(19) United States
(12) Patent Application Publication
(10) Pub. No.: US 2003/0125254 A1
(43) Pub. Date: Jul. 3, 2003

(54) Method of inhibiting smooth muscle proliferation

(75) Inventors: Walter J. Koch, Durham, NC (US); Jeffrey H. Lawson, Durham, NC (US)

Correspondence Address: NIXON & VANDERHYE, PC, 1100 N GLEBE ROAD, 8TH FLOOR, ARLINGTON, VA, 22201-4714, US

(73) Assignee: DUKE UNIVERSITY

(21) Appl. No.: 10/262,661

(22) Filed: Oct. 2, 2002

Related U.S. Application Data
(63) Continuation-in-part of application No. 09/400,861, filed on Sep. 21, 1999.
Application No. 09/400,861 is a continuation of application No. 08/943,208, filed on Oct. 3, 1997, now Pat. No. 5,981,487.

(51) Int. Cl. A61K 48/00; A61K 38/17

(52) U.S. Cl. 514/12; 514/44; 424/93.2

(57) ABSTRACT

The present invention relates, in general, to vascular smooth muscle proliferation and, in particular, to a method of inhibiting arterial and venous smooth muscle proliferation resulting, for example, from arterial injury, vein grafting or shunt implantation. The invention also relates to an expression construct encoding a Gβγ inhibitor suitable for use in such a method.

This application is a continuation-in-part of application Ser. No. 09/400,861, filed Sep. 21, 1999, which is a continuation of application Ser. No. 08/943,208, filed Oct. 3, 1997, now U.S. Pat. No. 5,981,487.

### TECHNICAL FIELD

The present invention relates, in general, to vascular smooth muscle proliferation and, in particular, to a method of inhibiting arterial and venous smooth muscle proliferation resulting, for example, from arterial injury, vein grafting or implantation of a synthetic conduit (e.g., a shunt). The invention also relates to an expression construct encoding a Gβγ inhibitor suitable for use in such a method.

### BACKGROUND

Several growth factors that induce cellular mitogenesis and proliferation act through membrane-embedded G protein-coupled receptors (GPCRs). GPCRs couple to, and stimulate, heterotrimeric G proteins which, upon activation, dissociate to Gα and Gβγ subunits. Both these molecules can transduce intracellular signals via activation of specific effector proteins. The intracellular signaling events leading to cellular proliferation following GPCR-activation appear to be transduced largely through the activation of p21^{ras} (Ras) and subsequent activation of the p42 and p44 mitogen-activated protein (MAP) kinases. Growth factors which act through GPCRs, such as lysophosphatidic acid (LPA) via the LPA receptor and norepinephrine via α2-adrenergic receptors, have been shown to activate Ras and MAP kinase primarily through Gβγ (Koch et al, Proc. Natl. Acad. Sci. USA 91:12706 (1994)).

The last 194 amino acids (Gly⁴⁹⁵-Leu⁶⁸⁹) of the bovine β-adrenergic receptor kinase-1 (βARK-1) represent a specific and selective Gβγ-inhibitor (see FIG. 1 for amino acid sequence of βARK-1-(495-689) and a nucleic acid sequence encoding same). βARK-1 is a Gβγ-dependent, cytosolic enzyme which must translocate to the membrane where it can phosphorylate its receptor substrate by physically binding to the membrane-anchored Gβγ (Pitcher et al, Science 257:1264 (1992)). The peptide encoded by the plasmid designated βARK-1-(495-689) Minigene (which peptide is designated βARK-1) contains the specific Gβγ-binding domain of βARK-1 (Koch et al, J. Biol. Chem. 268:8256 (1993)). When cells are transfected with the βARK-1-(495-689) Minigene (that is, the βARK_{CT} Minigene), or peptides containing the Gβγ-binding domain of βARK-1 are introduced into cells, several Gβγ-dependent processes are markedly attenuated including βARK-1-mediated olfactory receptor desensitization (Boekhoff et al, J. Biol. Chem. 269:37 (1994)), phospholipase C-β activation (Koch et al, J. Biol. Chem. 269:6193 (1994)) and Gβγ-dependent activation of Type II adenylyl cyclase (Koch et al, Biol. Chem. 269:37 (1994)). These studies demonstrate that the βARK-1-(495-689) peptide (that is, βARK_{CT}) is Gβγ-specific, that is, that it does not alter Gα-mediated responses (Koch et al, Proc. Natl. Acad. Sci. USA 91:12706 (1994); Koch et al, Biol. Chem. 269:37 (1994)). A further study utilizing the βARK_{CT} Minigene has demonstrated that the growth factor IGF-1, by binding to its specific receptor, activates the Ras-MAP kinase pathway via Gβγ. These results indicate that certain receptor-tyrosine kinase-mediated cascades include a Gβγ component, as do those for LPA and other agonists that activate classical GPCRs (Luttrell et al, J. Biol. Chem. 270:16495 (1995)).

The present invention is based, at least in part, on the observation that the βARK_{CT} peptide mediates inhibition of Gβγ function in vivo and that, in smooth muscle cells, that inhibition is associated with a modulation of cell proliferation.

### OBJECTS AND SUMMARY OF THE INVENTION

It is a general object of the invention to provide a method of inhibiting smooth muscle proliferation.

It is a specific object of the invention to provide a method of inhibiting uncontrolled smooth muscle cell proliferation by inhibiting Gβγ-signaling.

It is another object of the invention to provide a method of reducing intimal hyperplasia following vein grafting or implantation of a synthetic conduit and restenosis following arterial injury.

The foregoing objects are met by the method of the present invention which comprises introducing into smooth muscle cells at a body site an agent that inhibits Gβγ-mediated processes and thereby inhibits proliferation of the muscle cells. In one embodiment, the agent comprises a nucleic acid encoding a polypeptide corresponding to the Gβγ-binding domain of βARK. In accordance with this embodiment, the nucleic acid is introduced into the cells in a manner such that the polypeptide is produced and proliferation of the smooth muscle cells is inhibited.

Further objects and advantages of the invention will be clear from the description that follows.

### BRIEF DESCRIPTION OF THE DRAWINGS

FIG. 1. Amino acid sequence of βARK_{CT} (that is, βARK-1-(495-689)) polypeptide and nucleic acid sequence encoding same.

FIG. 2. RT PCR results from 3 day vein grafts treated with empty pRK5 and pRK βARK_{CT}. Lane 1 ΦX174HaeIII digested DNA markers with 2 of the size marker positions listed at the left; lanes 2 and 3, two control vein grafts transfected with pRK5 (plasmid); lanes 4 and 5, two vein grafts transfected with pRK βARK_{CT}; lane 6 negative control for PCR; lane 7, amplification of the positive control pRK βARK_{CT} purified plasmid. This gel displays two of each of the four 3 day vein grafts tested by RT PCR for transgene expression.

FIG. 3. MAP kinase activity in cultured vascular smooth muscle cells.

FIG. 4. Intima-to-media thickness ratio in rat carotid 28 days after balloon injury.

FIG. 5. Venous segment stained with X-Gal showing positive β-Gal transgene expression following delivery of 5×10¹¹ tvp of Adeno-βGal.

FIG. 6. Agarose gel of various pig samples and controls. The positive control for the RT-PCR reaction is the βARKct plasmid lane. The only other sample that is positive for this specific band is the Adeno-βARKct treated vein1. This gel shows that βARKct transgene delivery by incubating the clamped venous segment with 5×10¹¹ tvp Adeno-βARKct is specific for the vein as there is no transgene expression in the liver or lung and likewise in the EV-treated vein.

FIGS. 7A and 7B. Preliminary data using Adeno-βARKct in porcine Gore-tex AV fistulas. (FIG. 7A) Representative venous outflow stained sections taken 7 days after Gore-tex AV shunt surgery in pigs. Shown is (left) a control EV-treated vein with significant intimal hyperplasia and a section from an Adeno-βARKct treated vein (right). (FIG. 7B) The measured intimal and medial areas form n=4 each of 7 day Gore-tex grafts between control (PBS) treated, EV-treated (5×10¹¹ tvp) and Adeno-βARKct (5×10¹¹ tvp) treated venous outflow tracts. Data were analyzed using Stainpoint 1.1.4 software. *, p<0.05 vs controls (ANOVA).

FIG. 8. Survival curves showing AV Gore-tex graft patency in pigs in which the venous outflow tract was treated with PBS, EV or Adeno-βARKct. The βARKct treated grafts were 100% patent at this time-point and in the two control groups only 1 of 8 grafts was open at 28 days.

FIGS. 9A and 9B. Medial and intimal area in PBS, EV and Adeno-βARKct treated outflow tracts of Gore-tex AV shunts in pigs (n=4 each at 7 days (FIG. 9A) and n=4 each at 28 days (FIG. 9B)). Data were analyzed using Stainpoint 1.1.4 software. The βARKct significantly decreased both medial and intimal areas in both time-points *, p<0.05 vs controls (ANOVA).

### DETAILED DESCRIPTION OF THE INVENTION

Smooth muscle proliferation is problematic in several clinical settings including intimal hyperplasia following vein grafting (Davies and Hagen, Br. J. Surg. 81:1254 (1994)), shunt implantation (Schwab et al, Kidney Int. 56:1 (1999)) and restenosis following arterial angioplasty (Epstein et al, J. Am. Coll. Cardiol. 23:1278 (1994); French et al, Circulation 90:2402 (1994)). Smooth muscle cell proliferation is also associated with the development of atherosclerotic lesions (Katsuda et al, Amer. J. Pathol. 142:1787 (1993)). Smooth muscle cell proliferation can also be a problem when it occurs in the airways (Schramm et al, Life Sci. 59:PL9 (1996)), for example, in asthmatic patients and in individuals with idiopathic pulmonary fibrosis (Kanematsu et al, Chest 105:339 (1994)). The present invention provides a method of controlling smooth muscle proliferation in such settings by inhibiting Gβγ-dependent processes.

More specifically, the present invention provides a method of inhibiting smooth muscle proliferation at a body site comprising introducing into smooth muscle cells at the site an agent that effects inhibition of Gβγ-mediated processes. In one embodiment, the agent is a nucleic acid sequence that encodes a polypeptide that specifically inhibits Gβγ-dependent processes. One such agent is a nucleic acid encoding the Gβγ-binding domain of βARK.

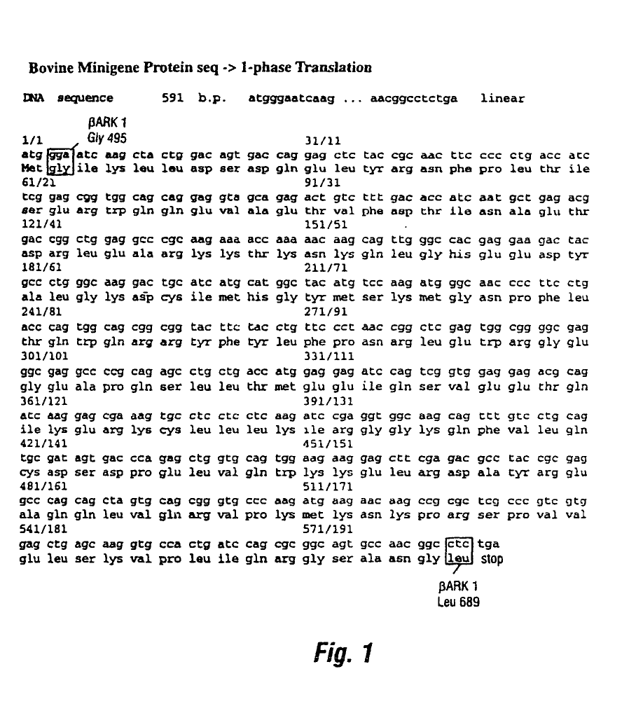

As one example, the present invention relates to a nucleic acid that encodes the last 194 amino acids of βARK-1, e.g., the amino acid sequence given in FIG. 1. Inhibitory portions of this polypeptide can also be used, for example, the 125 amino acid portion from position 546-670 of the FIG. 1 sequence or the 28 amino acid portion from position 643-670 of the FIG. 1 sequence. Methods that can be used to identify βARK (1 and 2) fragments that inhibit Gβγ-dependent processes are described by Koch et al, J. Biol. Chem. 268:8256 (1993) (see also Touhara et al, J. Biol. Chem. 270:17000 (1995); Inglese et al, Proc. Natl. Acad. Sci USA 91:3637 (1994); Luttrell et al, J. Biol. Chem. 270:16495 (1995); Hawes et al, J. Biol. Chem. 270:17148 (1995); Koch et al, Proc. Natl. Acad. Sci. USA 91:12706 (1994)). In one aspect of this example, the nucleic acid has the sequence also given in FIG. 1. Additionally, nucleic acids suitable for use in the present invention include those encoding functional equivalents of the polypeptide shown in FIG. 1, and portions thereof, that is, polypeptides that specifically inhibit binding of βARK to Gβγ.

In addition to the βARK fragments described above, fragments of the 33 Kda Gβγ-binding retinal phosphoprotein, phosducin, can also be used. Examples of fragments of phosducin suitable for use in the present invention, and methods of selecting same, are described by Xu et al, Proc. Natl. Acad. Sci. USA 92:2086 (1995) and Hawes et al, J. Biol. Chem. 269:29825 (1994). Suitable nucleic acid sequences encoding these peptides will be apparent to one skilled in the art.

In accordance with the present invention, the nucleic acid described above can be present in a recombinant molecule which can be constructed using standard methodologies. The recombinant molecule comprises a vector and the nucleic acid encoding the inhibitor. Vectors suitable for use in the present invention include plasmid and viral vectors. Plasmid vectors into which the nucleic acid can be cloned include any plasmid compatible with introduction into smooth muscle cells. Such vectors include mammalian vectors such as pRK5. Viral vectors into which the nucleic acid can be introduced include adenoviral vectors (see Examples II-IV), retroviral vectors (e.g., lentiviral vectors), and adenoassociated viral vectors, and combinations or derivatives thereof. The nucleic acid of the invention can be present in the vector operably linked to regulatory elements, for example, a promoter (e.g., a tissue specific or inducible promoter). Suitable promoters include, but are not limited to, the CMV, TK and SV40 promoters. Smooth muscle cell specific promoters can also be used, for example, an αSM22 promoter (see Moessler et al, Develop. 122:2415 (1996)). The nucleic acid of the invention can be present in a viral capsid.

In another embodiment of the present invention, a Gβγ inhibitor can be introduced directly into smooth muscle cells at a target site using methodologies known in the art. One such inhibitor is the polypeptide corresponding to the Gβγ-binding domain of LARK, for example, amino acids Gly⁴⁹⁵-Leu⁶⁸⁹ of βARK-1. Other suitable peptides of both βARK and phosducin are described above as are references disclosing methods suitable for use in selecting inhibitory peptides. The Gβγ inhibitor can be introduced into the target cells in a form substantially free of any proteins with which it may normally be associated. Polypeptide inhibitors can be produced recombinantly using the nucleic acid described above or chemically using known methods.

Compositions

The present invention also relates to pharmaceutically acceptable compositions comprising the nucleic acid or polypeptide of the invention. Such compositions can include, as active agent, the inhibitor or inhibitor-encoding sequence, in combination with a pharmaceutically acceptable carrier (e.g., water, phosphate buffered saline, etc.). The nucleic acid or polypeptide of the invention can also be present in a tissue adhesive or sealant (see, for example, U.S. Pat. No. 6,410,260, U.S. Pat. No. 5,290,552, WO93/05067 and Sierra, D. H., J. Biomat. App. 7:309 (1993)). The amount of active agent present in the composition can vary with the inhibitor or encoding sequence, the delivery system (in the case of a nucleic acid), the patient and the effect sought. Likewise, the dosing regimen can vary depending, for example, on the delivery system (particularly when a nucleic acid is used), the composition and the patient.

Therapy:

The present invention relates to the use in gene therapy regimens of a nucleic acid (eg a DNA sequence) encoding a Gβγ inhibitor, for example, a polypeptide corresponding to the βARK Gβγ-binding domain, or portions thereof as defined above.

Delivery of the nucleic acid of the invention can be effected using any of a variety of methodologies, including transfection with a plasmid or viral vector, such as those described above (see, for example, Steg et al, Circulation 90:1640 (1994), Guzman et al, Circulation 88:2838 (1993), Lee et al, Circulation Res. 73:797 (1993) and Plautz et al, Circulation 83:578 (1991)), or fusion with a lipid (eg a liposome) (see Takeshita et al, J. Clin. Invest. 93:652 (1994), Chapman et al, Cir. Res. 71:27 (1992), LeClerc et al, J. Clin. Invest. 90:936 (1992) and Nabel et al, Human Genet. 3:649 (1992)). Upon introduction into target cells, the nucleic acid is expressed and the Gβγ inhibitor is thereby produced.

Target cells include smooth muscle cells present, for example, in veins, arteries or airways. The target cells can be present, for example, at an anastomotic junction of a vascular fistula (e.g., an arteriovenous fistula) or vascular graft or shunt (e.g., an arteriovenous (AV) shunt). Introduction of the nucleic acid into the target cells can be carried out using a variety of techniques.

In the case of vein grafting, the techniques set forth in Examples I and II that follow can be used. As described in Example I, prior to grafting, the vein graft can be contacted with a solution containing the nucleic acid encoding the Gβγ inhibitor. While in Example I the nucleic acid is present in an plasmid, other systems can be used to effect delivery, including those described above and in Example II.

Alternatively, naked nucleic acid (e.g., naked DNA) present in a pharmaceutically acceptable carrier can be used. In accordance with the present method, the graft is held in contact with the nucleic acid for a period of time (e.g., 20-30 minutes) sufficient to permit introduction of the nucleic acid into smooth muscle cells of the graft and under conditions that facilitate the introduction of the nucleic acid without unacceptably compromising viability of the graft. Optimum conditions can readily be determined by one skilled in the art (see Examples I and II below).

Intimal hyperplasia of vascular smooth muscle cells at an anastomotic junction of an arteriovenous fistula or at an implantation site of a synthetic conduit (e.g. a shunt) can be inhibited by introducing a nucleic acid encoding the Gβγ inhibitor using techniques such as that described in Example IV. As described in Example IV, a vein segment to which a vessel (e.g., an artery) or conduit is to be attached can be isolated (e.g., by clamping) and then contacted with a solution containing nucleic acid encoding a Gβγ inhibitor. While in Example IV the nucleic acid is present in an adenovirus, other systems can be used to effect delivery, including those described above and in Example I and II. Alternatively, naked nucleic acid (e.g., naked DNA) present in a pharmaceutically acceptable carrier can be used. The vein segment is held in contact with the nucleic acid for a period of time (e.g., 20-30 minutes) sufficient to permit introduction of the nucleic acid into smooth muscle cells of the vein and under conditions that facilitate the introduction of the nucleic acid without unacceptably compromising the integrity of the vein. Optimum conditions can readily be determined by one skilled in the art. In addition, the nucleic acid (or inhibitory polypeptide) can be formulated in a tissue adhesive or sealant and applied, for example, to the anastomotic junction in an amount and under conditions such that intimal hyperplasia of vascular smooth muscle cells is inhibited.

Hemodialysis requires reliable access to the circulation, a common form of access being a polytetrafluoroethylene bridge graft (PTFE/Gore-tex). The patency rates of such grafts (shunts) are poor, the most common failure being thrombosis secondary to vascular smooth muscle neointimal proliferation. This occurs primarily in the area of the venous segment just proximal to the anastamosis (Schwab et al, Kidney Int. 56:1 (1999)). It is thus at this site (that is, at the venous outflow tract of the shunt or fistula) that nucleic acid encoding a Gβγ inhibitor is advantageously introduced.

In the case of arterial smooth muscle cells, the nucleic acid, advantageously in a viral vector, can be administered to an actual injury site (including an atherosclerotic site) via a catheter, for example, a balloon catheter. In accordance with this approach, inhibition of restenosis following angioplasty can be effected as can inhibition of smooth muscle cell proliferation at other arterial injury (or atherosclerotic) sites. (See Example III.) Catheters can also be used to deliver an inhibitory polypeptide or encoding nucleic acid to a stenosis, to an anastomotic junction or a point distal thereto.

As indicated above, other target sites include airway smooth muscle cells. Nucleic acids of the invention can be delivered to such cells, for example, in a viral vector, via aerosol administration. Optimum conditions can be readily determined by one skilled in the art.

As indicated above, the invention encompasses the administration of inhibitory polypeptides as well as nucleic acids encoding same. The polypeptides can be formulated in any of a variety of manners that facilitate incorporation into cells at the target site. Ideally, the polypeptides are relatively small molecules (e.g., about 27-28 amino acids in length, or less). Protein delivery approaches known in the art are suitable for use in the present invention (see, for example, Sternson, Ann. NY Acad. Sci. 507:19-21 (1987); Chen et al, Chem. Biol. 8:1123 (2001) and references cited therein).

The therapeutic methodologies described herein are applicable to both humans and non-human mammals.

It will be appreciated from a reading of this disclosure that the present invention makes possible a variety of studies targeting G protein pathways. Further therapeutic modalities can be expected to result from such studies.

Screening

The demonstration that βARK_{CT} inhibits smooth muscle cell proliferation makes possible assays that can be used to identify other smooth muscle cell proliferation inhibitors. For example, compounds to be tested for their ability to inhibit smooth muscle cell proliferation can be contacted with a solution containing Gβγ (eg purified Gβγ) and βARK, or a Gβγ binding portion thereof (eg purified βARK, or portion thereof), under conditions such that binding of Gβγ and βARK, or binding portion thereof, can occur. Test compounds that inhibit that binding can be expected to inhibit smooth muscle cell proliferation. Such tests compounds can also be screened for their ability to inhibit smooth muscle cell proliferation by determining the effect of the presence of the compound on Gβγ activation of βARK (eg using standard methodologies). A test compound that inhibits kinase activation can be expected to be suitable for use as an inhibitor of smooth muscle cell proliferation. Test compounds can also be screened by contacting cells (eg smooth muscle cells or fibroblasts) with such a compound and determining the effect of the test compound on LPA dependent activation of MAP kinase. A test compound that inhibits such activation can be expected to inhibit smooth muscle cell proliferation.

Certain aspects of the present invention are described in greater detail in the non-limiting Examples that follow.

### EXAMPLE I

Effect of βARK_{CT} on the Formation of Vein Graft Intimal Hyperplasia and Phenotypical Functional Alterations

Experimental design: Forty New Zealand White rabbits underwent carotid interposition vein bypass grafting. Prior to grafting, veins were incubated in heparinized Ringer's lactate (controls; n=18), or plasmid solutions containing either βARK_{CT} (n=14; 190 μg/ml) or empty plasmid DNA (plasmid: n=8; 190 μg/ml) for 30 mins at 37° C. Twenty-four vein grafts (n=10 controls, n=6 plasmid, n=8 βARK_{CT}) were harvested at 28 days by perfusion fixation. Intimal and medial dimensions of vein grafts were calculated by videomorphometry. Sections were taken for scanning and transmission electron microscopy (TEM). Ten vein grafts (n=5; control and βARK_{CT}) were analyzed for in vitro contractile responses to norepinephrine and serotonin in the presence and absence of pertussis toxin (PTx) to categorize receptor G-protein receptor coupling. Six vein grafts (n=3; control and βARK_{CT}) were harvested at 3 days for βARK-1 protein and mRNA (RT-PCR) expression.

Transgene constructs: Gene transfer to the experimental vein grafts was done utilizing the previously described plasmid which contains cDNA encoding the last 194 amino acid residues (Met-Gly⁴⁹⁵-LeU⁶⁸⁹) of bovine βARK_{CT} (pRK-βARK_{CT}) (Koch et al, Proc. Natl. Acad. Sci. USA 91:12706 (1994); Koch et al, J. Biol. Chem. 268:8256 (1993)). This peptide contains the experimentally determined (Gln⁵⁴⁶-Ser⁶⁷⁰) Gβγ binding domain. The empty pRK5 plasmid was used as the negative control as previously described (Koch et al, Proc. Natl. Acad. Sci. USA 91:12706 (1994); Koch et al, J. Biol. Bhem. 269:6193 (1994)). Large scale plasmid preparations of pRK5 and pRK βARK_{CT} were purified using Qiagen columns (Qiagen Inc., Chatsworth, Calif.) prior to vein graft gene transfer.

Analysis of βARK_{CT} transgene expression: Three day vein grafts were utilized for analysis of specific transgene expression. βARK_{CT} mRNA expression was determined by standard methods of reverse transcriptase-polymerase chain reaction (RT-PCR) (Ungerer et al, Circularion 87:454 (1993)) using a RT-PCR kit utilizing TaqPlus DNA Polymerase (Stratagene Inc. La Jolla, Calif.). Total RNA was first isolated using the single step reagent RNAzol (Biotecx Inc., Houston, Tex.) (Chomezynski et al, Anal. Biochem. 161:156 (1987))) and treated with DNase I to eliminate any possible plasmid contamination. A βARK_{CT} primer set was utilized to specifically amplify βARK_{CT} mRNA. The primers utilized were as follows: sense primer (corresponding to the start of βARK_{CT}) 5′-GAATTCGCCGCCACCATGGG-3′; antisense primer (corresponding to the β-globin untranslated region linked to the end of the βARK_{CT} cDNA (Koch et al, J. Biol. Chem. 269:6193 (1994)) 5′-GGAACAAAGGAACCTTTAATAG-3′. This primer set amplifies a 670 base pair fragment corresponding to βARK_{CT} mRNA.

Operative Procedure: Anesthesia was induced and maintained with subcutaneously injected ketamine hydrochloride (60 mg/kg, Ketaset, Bristol Laboratories, Syracuse, N.Y.) and xylazine (6 mg/kg, Anased, Lloyd Laboratories, Shenandoah, Iowa.). Antibiotic prophylaxis with 30,000 IU/kg of benzanthine and procaine penicillin (Durapen, Vedco Inc., Overland Park, Kans.) was given intramuscularly at the time of induction. Surgery was performed using an operating microscope (JKH 1402, Edward Weck Inc., Research Triangle Park, N.C.) under sterile conditions. After exposure through a midline longitudinal neck incision, the right external jugular vein was identified, its branches were diathermied at a distance from the vein to minimize injury and it was then dissected out. Following excision, the vein was kept moist in a heparinized Ringer lactate solution (5 IU/ml, Heparin, Elkins-Sinn Inc., Cherry Hill, N.J.) for approximately 15 minutes while the right common carotid artery was identified, dissected and both proximal and dismal control obtained. Heparin (200 IU/kg) was administered intravenously. A proximal longitudinal arteriotomy was made and one end of the reversed jugular vein was anastomosed to the artery in an end-to-side manner using continuous 10-O microvascular monofilament nylon suture (Ethilon, Ethicon Inc., Somerville, N.J.). The distal anastomosis was performed in a similar manner. Throughout the procedure, care was taken to avoid unnecessary instrumentation of the vein graft. The right common carotid was ligated and divided between the two anastomoses with 4-O silk sutures and the wound closed in layers.

Morphology: Three vein grafts were harvested 28 days after surgery. Following isolation and systemic heparinization (200 IU/kg, i.v.), the vein grafts were perfusion fixed in situ at 80 mmHg with an initial infusion of Hanks Balanced Salt Solution (HBSS, Gibco Laboratories, Life Technologies Inc., Grand Island, N.Y.) followed by 2% glutaraldehyde made up in 0.1 M cacodylate buffer (pH 7.2) supplemented with 0.1 M sucrose to give an osmolality of approximately 300 mOsm. After 60 minutes, the specimen was removed, immersed in the glutaraldehyde fixative for a further 24 hours. Cross-sections from the mid-portion of the vein graft were processed for light microscopy. Following standard histological procedures, each specimen was stained with a modified Masson's trichrome and Verhoeff's elastin stain and dimensional analysis was performed by videomorphometry (Innovision 150, American Innovision Inc., San Diego, Calif.). The intima and media were delineated by identification of the demarcation between the criss-cross orientation of the intimal hyperplastic smooth muscle cells and circular smooth muscle cells of the media and the outer limit of the media was defined by the interface between the circular smooth muscle cells of the media and the connective tissue of the adventida. The thickness of each layer was also determined. A ratio of the intimal and medial areas (intimal ratio=intimal area/[intimal+medial areas]) and a luminal diameter to cross-sectional wall thickness (luminal index=luminal diameter/[cross-sectional wall thickness]) was calculated.

In vitro contractile studies: Under anesthesia, the original incision was re-opened and the jugular vein and vein graft isolated. The midpart of each vessel was sectioned in situ into two 5 mm segments and excised. These rings were suspended immediately from two stainless steel hooks in 5 ml organ baths containing oxygenated Krebs solution (122 mM NaCl, 4.7 mM KCl, 1.2 mM MgCl₂, 2.5 mM CaCl₂, 15.4 mM NaHCO₃, 1.2 mM KH₂PO₄ and 5.5 mM glucose; maintained at 37° C. and bubbled with a mixture of 95%) O₂ and 5% CO₂). One hook was fixed to the bottom of the bath and the other was connected to a force transducer (Myograph F-60, Narco Bio-Systems, Houston, Tex.). The isometric responses of the tissue were recorded on a multichannel polygraph (Physiograph Mk111-S, Narco Bio-Systems, Houston, Tex.). The tissues were then placed under 0.5 grams tension and allowed to equilibrate in physiologic Krebs solution for one hour. During the equilibration period, the Krebs solution was replaced every 15 minutes. Following equilibration, the resting tension was adjusted in 0.25 gram increments from 0.25 to 2.5 gram and the maximal response to a modified oxygenated Krebs solution (60 mM KCl, 66.7 mM NaCl, 1.2 mM MgCl₂, 2.5 mM CaCl₂, 15.4 mM NaHCO₃, 1.2 mM KH₂PO₄ and 5.5 mM glucose) was measured at each resting tension to establish a length-tension relationship. Based on these results, the optimal resting tension for each ring (the tension at which the response to the modified Krebs solution was maximal) was determined and the ring was set at this tension for subsequent studies. Norepinephrine (10⁻⁹ to 10⁻⁴M) was added cumulatively in half molar increments and the isometric tension developed by the tissue was measured. After washout and re-equilibration, dose response curves were obtained for serotonin (10⁻⁹ to 10⁻⁴M). The responses to each agonist were assessed with and without the presence of PTx (100 ng/ml pre-incubated for 60 minutes) (Davies et al, J. Clin. Invest. 94:1680 (1994)). All compounds were obtained from Sigma Chemical Company (St. Louis, Mo.).

Data and Statistical Analysis: The EC₅₀ value, the concentration for the half maximal response, for each agonist in each ring was calculated by logistic analysis and is expressed as log₁₀ [EC₅₀] (Finney, Statistical methods in biological assay. London: Charles Griffin, pp. 349-369 (1978)). All data are presented as the mean±standard error of the mean (s.e.m.) and statistical differences between groups were tested by ANOVA with post hoc Tukey-Kramer multiple comparison tests for the functional studies and with a Kruskal-Wallis nonparametric ANOVA with post hoc Dunn's multiple comparison tests for the morphometric data.

Results

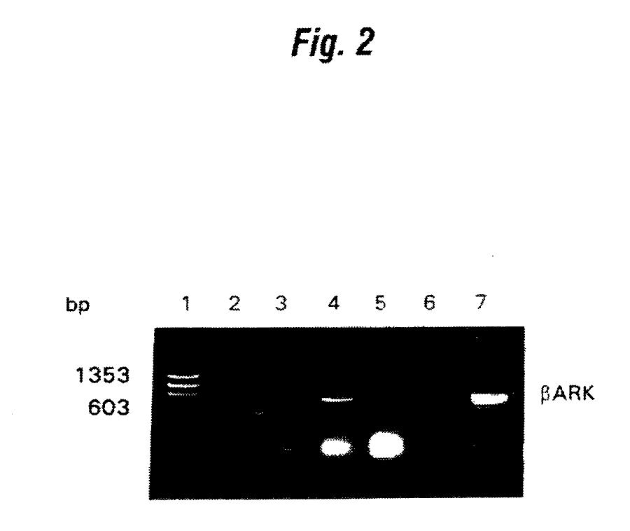

Transgene expression: Successful transfection of the vein grafts was demonstrable at three days after surgery. βARK_{CT} mRNA was specifically amplified from DNase I treated total RNA using RT-PCR from vein grafts treated with pRK-βARK_{CT} while control grafts treated with the empty pRK5 plasmid showed no transgene expression (FIG. 2). Since the amount of tissue available is small, protein immunoblotting for βARK_{CT} peptide expression was not possible.

Intimal hyperplasia: All animals survived to 28 days, and all grafts were patent at harvest. Microscopically, the luminal surfaces of the vein grafts from each group were covered by a layer of intact endothelial cells, beneath which lay a hyperplastic intima with the smooth muscle cells of the intimal hyperplasia arranged in a crisscross pattern with little extracellular matrix. The medial smooth muscle cells in the grafts from each group appeared slender, were arranged in a circular pattern, and contained a greater amount of extracellular matrix suggestive of medial hypertrophy. At 28 days, there was a significant 37% reduction in intimal thickness in βARK_{CT} vein grafts (45±4 μm) compared to either plasmid (69±3 μm) or control (70±4 μm) vein grafts without a significant change in medial thickness (70±4 μm, 65±5 μm and 77±3 μm, respectively). Dimensional analysis of the control and treated groups is shown in Table I. There was a 52% decrease in intimal area (Table I) while the medial area was unchanged in the βARK_{CT} compared to the plasmid treated vein grafts (Table I). The intimal ratio was significantly reduced in the βARK_{CT} vein grafts (p<0.01; 0.36±0.02, mean±s.e.m.) compared to either plasmid (0.54±0.02) or control vein grafts (0.52±0.02). The luminal area of the βARK_{CT} treated vein grafts was 41% less than the plasmid treated vein grafts while the luminal indices were not significantly different for the control, plasmid and βARK_{CT} vein grafts.

TABLE I
Dimensional Analysis

| | Control | Plasmid | βARK_{CT} | p-value |
|---|---|---|---|---|
| Lumen (mm²) | 20.5 ± 1.5 | 28.6 ± 4.01 | 16.6 ± 2.33† | 0.02 |
| Intima (mm²) | 1.14 ± 0.09 | 1.29 ± 0.12 | 0.62 ± 0.03\ | 0.01 |
| Media (mm²) | 1.08 ± 0.11 | 1.29 ± 0.17 | 1.12 ± 0.10 | 0.18 |
| Intimal ratio | 0.52 ± 0.02 | 0.54 ± 0.02 | 0.36 ± 0.02* | 0.02 |
| Luminal Index | 39.4 ± 2.6 | 44.2 ± 3.1 | 37.8 ± 3.9 | 0.4 |
| The area of the lumen, the intimal and the medial layers from control, plasmid and βARK_{CT} treated grafts. The intima ratio (intimal area/ [intimal + | | | | |
| # medial areas]) and luminal index (luminal diameter/ (cross-sectional wall thickness]) are also shown. Values are the mean ± s.e.m. Statistical Analysis is by | | | | |
| # Kruskal-Wallis nonparametric ANOVA with post hoc Dunn's multiple comparison tests (p < 0.05 vs. Control; †p < 0.05 vs. Plasmid) | | | | |

Contractile function of experimental vein grafts: Control and βARK_{CT} treated vein grafts responded with concentration dependent contractions to the agonists norepinephrine and serotonin. In the presence of PTx at concentrations sufficient to produce 100% ADP ribosylation of G-proteins (Davies et al, J. Clin. Invest. 94:1680 (1994)), the contractile responses in control vein grafts to norepinephrine (p<0.01) and serotonin (p<0.01) were significantly reduced compared to untreated control vein grafts (Table II). This is the typical functional alteration seen in experimental vein grafts as native veins do not have a PTx sensitive component in their contractile responses to these G-protein coupled agonists. In contrast, the responses of the βARK_{CT} treated vein grafts to norepinephrine and serotonin were unchanged in the presence of PTx indicating the loss of a Gαᵢ component (Table II).

TABLE II
Sensitivity of Contractile Responses

| | | Norepinephrine | | |
|---|---|---|---|---|
| | Norepine- | with pertussis | | Serotonin with |
| | phrine | toxin | Serotonin | pertussis toxin |
| Control | 6.00 ± 0.09 | 5.16 ± 0.09* | 6.34 ± 0.10 | 5.54 ± 0.26* |
| βARK_{CT} | 5.91 ± 0.19 | 5.81 ± 0.18 | 6.57 ± 0.10 | 6.55 ± 0.13 |

Data are expressed s the mean ± s.e.m.. Contractile sensitivity is shown as −logEC₅₀. *p < 0.01 compared to corresponding pertussis toxin untreated vessel by ANOVA.

Electron microscopy of vein grafts: Scanning electron microscopy from both control vein grafts and vein graft transfected with empty plasmid showed the luminal surface to be lined with sharply outlined endothelial cells with well defined cell borders. Occasional junctional stomata were noted. Transmission electron micrograph of these vein grafts confirmed the presence of well formed endothelial cells, beneath which were well developed smooth muscle cells of both contractile (cytoplasm predominantly filled with contractile filaments) and synthetic phenotypes (cytoplasm filled with synthetic organelles) in a loose connective tissue matrix. No inflammatory cells or evidence for apoptosis was identified in these grafts. Scanning electron microscopy from vein grafts transfected with βARK_{CT} showed a similar picture to the control and plasmid transfected vein grafts with well preserved, normal appearing endothelial cells with occasional stomata at their junctions on the luminal surface. Transmission electron microscopy showed a similar ultrastructural pattern to the control and plasmid transfected vein grafts. One difference in the βARK_{CT} treated vein grafts was seen at higher magnification, which was the appearance of numerous cells with ultrastructural evidence of apoptosis with nuclear fragmentation, membrane disruption, and in places, disintegration products consisting of endoplasmic reticulum.

### EXAMPLE II

### Adenoviral Mediated Inhibition of Gβγ Signaling Limits Development of Intimal Hyperplasia

Thirty-seven male NZW rabbits had interposition bypass grafting of the carotid artery using the jugular vein. Prior to grafting, veins were incubated in heparinized Ringer's lactate (controls; n=10), solutions containing adenoviral vectors (1×10¹ PFU/ml) encoding βARK_{CT} (n=19), β-galactosidase (β-Gal; n=3), or empty vector (EV; n=3). (For details of adenoviral vector, see Drazner et al., J. Clin. Invest. 99:288 (1997).) After implantation, vein grafts were coated with 4 ml of 30% pluronic gel with or without the respective viral solutions (1.7×10⁹ PFU/ml).

The efficacy of βARK_{CT} transfection in vein grafts was verified by RT-PCR on days 3, 5 and 7 postoperatively (n=3 per time-point). To determine the cellular expression of the transfected gene, X-Gal staining for the marker gene β-Gal was performed on day 3. Positive (blue) cells were seen throughout the wall of the β-Gal vein grafts. At 28 days, the intimal thickness) in βARK_{CT} vein grafts (n=6) was reduced by 33% with no significant change in the medial thickness (MT), compared to control (n=6) and EV (n=3) grafts (Table III). Contractile studies showed enhanced sensitivity in response to norepinephrine (NE) and serotonin (5-HT) in 28 day βARK_{CT} vein grafts (n=4), as compared to controls (n=4) and EV (n=2), and insensitivity to pertussis toxin (PT) (Table III). Viral infection of vein grafts with EV did not alter vein grafts dimensions or contractility.

TABLE III

| | IT(μm) | MT(μm) | NE | NE + PT | 5-HT | 5-HT + PT |
|---|---|---|---|---|---|---|
| βARK_{CT} | 57 ± 4* | 68 ± 3 | 6.35 ± 0.06† | 5.92 ± 0.25 | 6.74 ± 0.10† | 6.46 ± 0.19 |
| EV | 86 ± 10 | 87 ± 4 | 5.67 ± 0.03 | — | 5.65 ± 0.08 | — |
| Control | 85 ± 4 | 91 ± 5 | 5.85 ± 0.10 | 5.17 ± 0.14‡ | 6.17 ± 0.10 | 5.32 ± 0.18‡ |

Data are shown as mean ± S.E.M. Sensitivity is defined as -logED₅₀. *p < 0.5 compared to EV and control (Kruskal-Wallis with post-hoc Dunn's test); †p < .01 compared to EV and control (ANOVA); ‡p < .001 compared to without PT (Student t-test).

The results demonstrate that inhibition of Gβγ signaling with adenoviral mediated βARK_{CT} in vivo transfection effectively modifies the structural and functional hyperplastic abnormalities in experimental vein grafts.

### EXAMPLE III

### Inhibition of Restenosis of Injured Carotid Artery with βARK_{CT} Adenovirus

The rat common carotid injury is a well studied and reliable model of neo-initimal cell proliferation (Clowes et al, Lab. Invest. 49:327 (1983)). Following the application of a high pressure vascular damage, vascular smooth muscle cells migrate from the tunica media through the basal lamina into the tunica intima, were they proliferate. Those mechanisms are sustained by growth factor released from cells infiltrating the neo-intima and other substances circulating in the blood stream. At the vascular smooth muscle cells level, those factors interact with specific receptors thus activating intracellular mechanisms of proliferation. Among them, mitogen activated protein (MAP) kinase plays a relevant role, being at the confluence of several receptor activated pathways. It has been demonstrated recently that the βγ subunit of the heterotrimeric G protein mediates the activation of the MAP kinase induced by Gi coupled receptors. The carboxyterminus portion of the G coupled receptor kinase βARK1 binds the βγ subunit, thus inhibiting its signaling on MAP kinase.

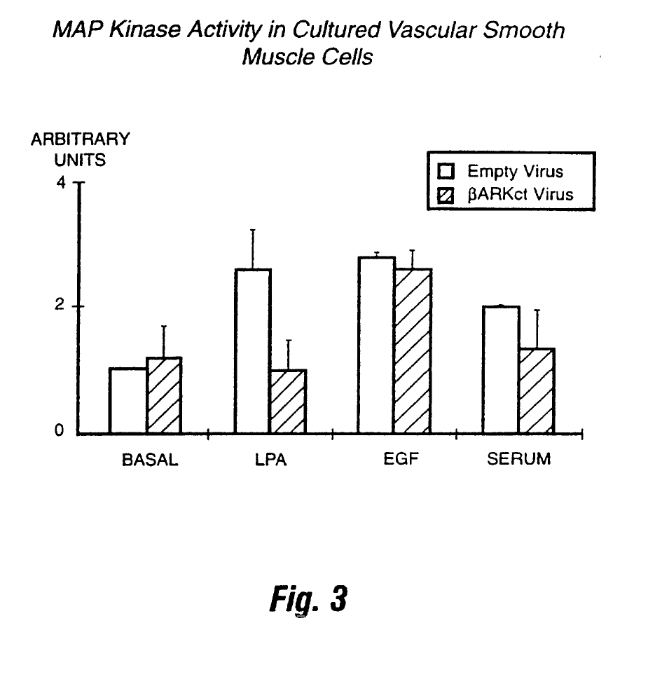

Using adenoviral mediated gene delivery (see Drazner et al., J. Clin. Invest. 99:288 (1997), it was possible to demonstrate that induction of expression of βARK_{CT} resulted in the inhibition of proliferation of vascular smooth muscle cells in the rat carotid injury model. Firstly, it was shown that in rabbit aortic smooth cells in culture (see Davies et al, J. Surg. Res. 63:128 (1996)), the virus was able to infect and replicate, resulting in the inhibition of the activation of MAP kinase in response to Gi coupled receptor stimulation. The lysophosphatidic receptor, a major mitogen circulating in the serum, was assessed. Furthermore, MAP kinase activation in response to fetal bovine serum and epidermal growth factor was assessed. βARK_{CT} adenovirus in the cultured vascular smooth muscle cells inhibited LPA (−58% of the same response observed in empty virus treated cells) and serum (−38%) activation of MAP kinase, without interfering with basal (+18%) and EGF (−7%) response (see FIG. 3).

The feasibility of infection of vascular smooth muscle cells in vivo was also determined using the rat common carotid after balloon injury. The balloon injury was performed through the external carotid in the common carotid by means of a Fogarty catheter with the balloon inflated at 1.5 atmospheres. After the injury, the virus (0.5×10¹⁰ PFU) was injected into the lumen of the common carotid through the external carotid and incubated for 30 min. The external carotid was then tied up by means of silk sutures and the blood flow in the common carotid was restored. A further dose of virus (−0.5×10¹⁰ PFU) was applied at the external of the common carotid by means of pluronic gel. The wound was closed in layers. A virus containing the bacterial gene LAC-Z encoding β-galactosidase was used, and after three days from the injury and the application of the virus, β-Gal staining was performed on cyo-fixed carotid arteries. The staining demonstrated that the application of the virus from the lumen and the external by means of the pluronic gel resulted in the infection of the arterial wall from the intima throughout the adventitia.

Successively, using the same protocol, it was determined whether the virus encoding the βARK_{CT} was able to replicate in the carotid. After five days from the injury and the application of the virus, RT-PCR was performed on DNAse treated RNA extracted from rat common carotids. This analysis allowed testing of the efficacy of the virus to replicate in vivo.

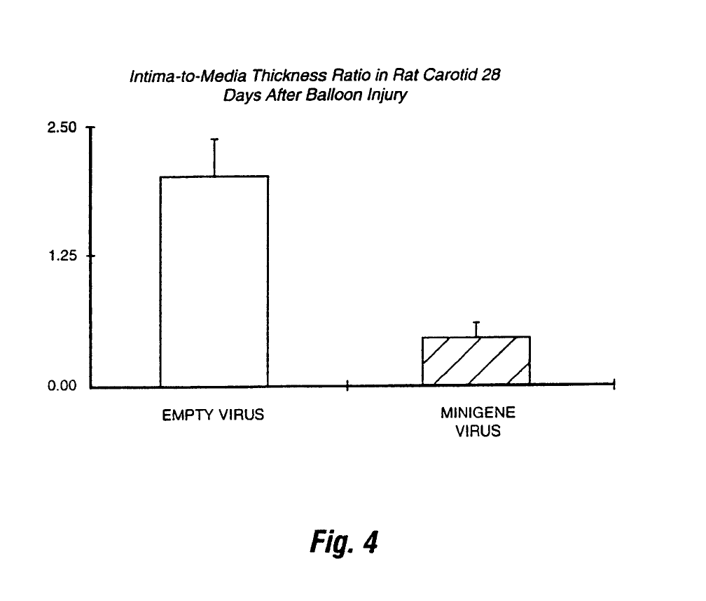

In a further set of experiments, injured common carotid was treated with βARK_{CT}, or empty virus. After 28 days, the carotids were harvested and fixed and analyzed for morphometric measurements. A intimal proliferation index was obtained by the intima-to-media thickness ratio. In animals treated with empty virus, the intima proliferation was 2.036±0.312, while in the βARK_{CT} treated carotid, this ratio was 0.426±0.137, significantly reduced as compared to the empty virus treatment (p<0.01) (see FIG. 4).

### EXAMPLE IV

Adenoviral Constructs:

The adenoviral backbone for Adv-βARKct is a second-generation replication-deficient serotype 2 adenovirus with deletions of E1 and E4 (except for ORF6) as previously described (White et al, Proc. Natl. Acad. Sci. USA 97:5428 (2000)). Aliquots of 5×10¹¹ tvp's (total viral particles) were thawed and mixed in 1.6% (v/v) heparin-PBS for a final volume of 2 ml immediately before intravascular delivery.

Animals:

70 lb. Yorkshire cross-bred swine were housed at the Duke University Vivarium. Animals were fed a regular diet and were pre-treated with 650 mg aspirin PO for two days before surgery. Animals were made NPO the day of surgery.

Surgical Protocol:

Animals were tranqualized with a ketamine-acepromazine-glycopyrrolate solution and sedated with 2.5% thiopental, intubated with a #6 endotracheal tube, and maintained on isofluorane for the duration of the procedure. Prior to skin incision, animals were given 1 g kefzol IV. The animal was placed in the supine position on the operating table and the neck prepared with betadine. The animal was then draped in a sterile fashion and a 15 cm longitudinal neck incision was made. The left common carotid artery was isolated first, followed by the right external jugular vein. An 8 cm segment of vein was freed from surrounding tissues and all tributaries off of the vein were ligated with 3-0 silk suture (Ethicon). The animal was then treated with 100 U/kg of heparin IV followed by 1,000 U/hr for the duration of the procedure. A 24 g IV catheter was inserted in the middle of the isolated right external jugular vein segment, secured using 6-0 prolene suture (US Surgical), and capped. The 8 cm vein segment was then clamped both distally and proximally and the blood was removed from the vein via the catheter. The vein segment was then washed three times with a 1.6% (v/v) heparin-saline solution to remove any residual blood. At this time, Adeno-βARKct, EV (control) or PBS (control) was administered in 2 mL of heparin-saline solution. Attention was then turned to the left carotid artery. The artery was clamped and a 7 mm arteriotomy performed. An oblique end-to-side anastomosis was performed between the artery and a 6 mm internal diameter PTFE graft (Atrium) using a running 6-0 prolene suture. Once fashioned, the arterial clamp was removed and the graft flushed with a heparin-saline solution. Good flow was observed through the artery and into the graft. The graft was then tunneled beneath the sternoclidomastoid muscles and brought into the proximity of the right external jugular vein. At this time (30-40 min), the viral or control solution was removed from the vein segment via the catheter, the catheter was removed, and a 7 mm venotomy was performed directly over the catheter injection site. The arteriovenous fistula was then completed with an oblique end-to-side anastomosis between the PTFE graft and the right external jugular vein, again using a running 6-0 prolene suture. All clamps were removed and good flow was observed through the graft. The left carotid artery distal to the PTFE anastomosis was then doubly tied off with 3-0 silk. Good hemostasis was achieved. The muscular and subcutaneous layers were closed in one layer with running 2-0 vicryl suture (Ethicon) and the skin closed with staples. Bactroban was applied over the wound and the pig was given 1 g cefazoline IM and 0.15 mg buprenorphine IM. Animals were maintained at the Duke University Vivarium during the post-operative period following IACUC protocols. Doses of 0.15 mg buprenorphine Im were used for post-operative pain management as needed. Animals were treated with 325 mg asprin PO QD post-operatively.

At the time of harvest, animals were tranquilized and sedated as described above. The old surgical incision was re-opened and the 8 cm segment of the right external jugular vein was isolated and freed from surrounding tissues.

β-Gal Staining:

Staining for β-galactosidase (β-Gal) expression was carried out as described previously (Iaccarino et al, Proc. Natl. Acad. Sci. USA 96:3945 (1999)). Briefly, the 8 cm right external jugular anastomosis was isolated and secured in situ using 3-0 silk ties. The PTFE graft was then divided, blood was removed from the venous anastomosis, and the venous tissue fixed with 2% formaldehyde and 0.2% gultaraldehyde in PBS, pH 7.2 at 100 mmHg for 5 min. The 8 cm segment was excised and incubated in the staining solution containing 5 mM K₄Fe(CN)₆, 5 mM K₃Fe(CN)₆, 2 mM MgCl₂, 0.02% (v/v) NP-40, 0.01% (w/v) sodiumdeoxycholate, and 1 mg/mL X-gal in PBS (pH 7.5) at 37° C. for 2 h. The artery was then placed in fixative and refrigerated for an additional 2 h. After fixation, the artery was embedded in paraffin and sectioned. The sections were counterstained with hematoxylin and eosin and the number of infected cells was counted under light microscopy. Infection efficiency was determined as the ratio of either infected cell number to the total number of cells or as the ratio of the area of the arterial wall stained blue to the total arterial wall area. The areas were determined using light microcopy connected to a CCD camera and a PC computer.

Histological Staining and Restenosis Measurements:

The 8 cm treated anastomosis segment of right external jugular vein were harvested at either 7 or 28 days post-operatively and perfusion-fixed with formalin. Venous segments were embedded in paraffin and cut in cross-section for histological staining and measurements. 5 micron cross-sections were taken every 100 microns and stained with Masson trichrome. At least 50 sections were obtained from each carotid, and the 5 sections with maximal hyperplasia were identified and measured. Digital images were taken of these sections and measured with StainPoint 1.14 software.

RNA Preparation and RT-PCR:

To assess in vivo βARKct transgene delivery to the venous outflow tract, a group of pigs (n=4) was sacrificed after 6 days and the right (experimental) and left (control) external jugular veins, liver, and lungs were harvested, rinsed in PBS, and frozen in liquid nitrogen. Total RNA was isolated using TRIzol reagent (Gibco). One microgram of total RNA was reverse transcribed into cDNA using MuLV reverse transcriptase by incubating reagents at room temperature for 10 min, followed by 15 min at 42° C. The cDNA products were then used as PCR templates for the amplification of a 600-bp βARKct fragment. Primer pairs were a sense primer, 5′-GAATTCGCCGCCACCATGGG-3′ (corresponding to βARKct), and an antisense primer, 5′-GGAACAAAGGAACCTTTAATAG-3′ (corresponding to the human β-globin sequence attached to the end of the βARKct (Iaccaino et al, Proc. Natl. Acad. Sci. USA 96:3945 (1999)). The PCR consisted of 35 cycles between 95° C. (15 sec) and 55° C. (45 sec). Controls included reactions without template, without reverse transcriptase, and water alone. Primers for glyceraldehydes phosphate dehydrogenase (GAPDH) (sense: 5′-GACCCCTTCATTGACCTCAAC-3′, antisense: 5′-CTTCTCCATGGTGGTGAAGA-3′) were used as controls. Reaction products were resolved on a 1.2% agarose gel and visualized using ethidium bromide.

Statistical Analysis:

Data are presented as mean +/−SE. In vivo histological findings of hyperplasia and the effects of PARKct treatment were analyzed by ANOVA.

A positive effect of the βARKct delivered ex vivo to rabbit vein-grafts either by a plasmid or adenovirus has been observed (Huynh et al, Surgery 124:177 (1998), Davies et al, Arterioscler. Thromb. Vasc. Biol. 18:1275 (1998)). In these studies, intimal hyperplasia seen in jugular vein segments grafted to the carotid circulation in rabbits at 28 days post-surgery/gene transfer was significantly inhibited by >30% in βARKct-treated grafts. To study an in vivo model of venous occlusion due to intimal hyperplasia after placement of a fistula or shunt, a porcine model has been used in which a carotid artery-to-jugular shunt is surgically implanted using a polytetrafluoroethylene (PTFE/Gore-tex) bridge graft (a common form of circulatory access for hemodialysis). To study an in vivo model of venous occlusion after placement of a graft due to intimal hyperplasia, a porcine model is used in which a carotid artery-to-jugular vein shunt is surgically implanted using polytetrafluoroethylene (PTFE/Gore-tex). This serves as a pre-clinical model to study the effectiveness and feasibility of molecular therapies to combat venous access failure in hemodialysis patients.

In the present studies, the Adeno-βARKct (5×10¹¹ total viral particles, tvp) is used, which is a replication-deficient adenovirus carrying the βARKct transgene (White et al, Proc. Natl. Acad. Sci. USA 97:5428 (2000)). In both a 7-day and a 28-day study, control animals received either saline (PBS) or an empty adenoviral vector with no transgene (EV). Gene delivery was targeted to the venous outflow tract of the Gore-tex AV shunts that were placed between the carotid arterial and jugular venous circulation of 70 lb pigs. The adenovirus (5×10¹¹ tvp) is incubated for approximately 30 min in the venous outflow tract via clamping of the segment to allow for slight pressurized conditions in this segment that may facilitate gene delivery. Initial studies also utilized an adenovirus containing the marker gene β-galactosidase (Adeno-βGal) in order to visualize gene delivery to the venous outflow tract segment. FIG. 5 displays a representative section of porcine jugular vein distal to the Gore-tex graft anastomosis 3 days after a 30 min incubation of Adeno-βGal and grafting.

For delivery of Adeno-βARKct, Reverse Transcriptase Polymerase Chain Reaction (RT-PCR) has been utilized in order to verify that the βARKct transgene is delivered to the venous outflow tract of Gore-Tex AV shunts in pigs. FIG. 6 shows an agarose gel displaying positive expression in a graft treated with 5×10¹¹ tvp Adeno-βARKct.

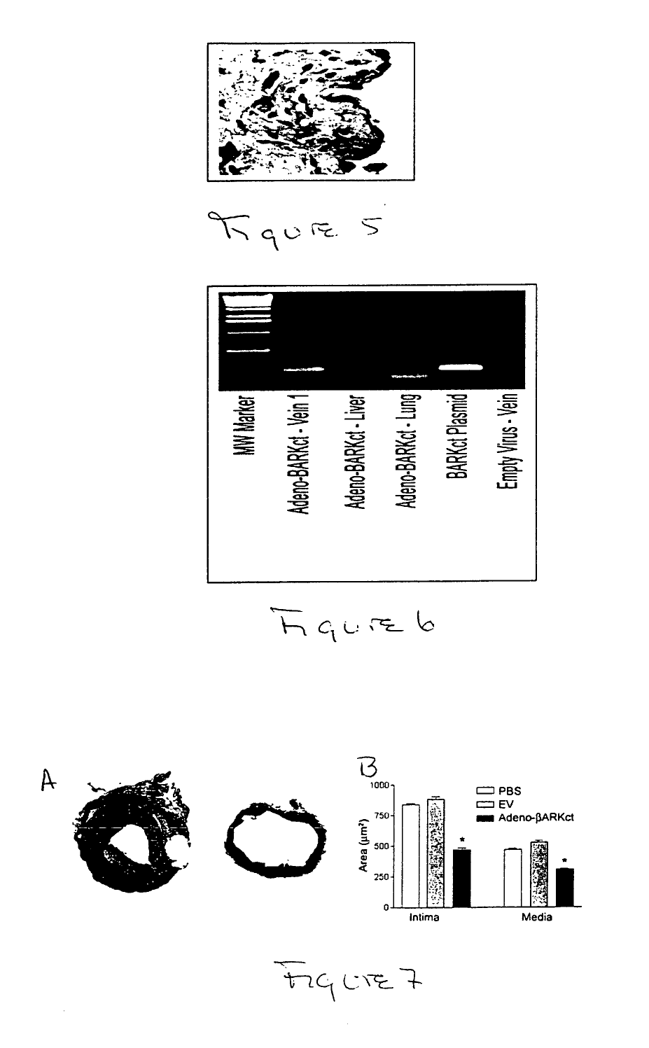

Using this model of Gore-tex AV shunting and adenoviral-mediated gene delivery, a initial study was carried out at one week examining medial and intimal thickening (i.e., hyperplasia), as well as a more complete study at 28 days, where graft patency has been examined as well as morphology and histology. In each study, the venous outflow tract was treated with 5×10¹¹ tvp of either Adeno-βARKct or EV or PBS alone as a second control (n=4 each). The treatment was administered in a blinded fashion and the treatment-status of individual pigs was not uncoded until completion of the study. Importantly, as shown in FIG. 7A, control un-treated shunts stimulate a robust venous intimal hyperplasia. Thus, this demonstrates that the model simulates the failure rates of human AV fistulas/shunts. In this 7-day study, the venous outflow tract was treated with either Adeno-βARKct (n=4), EV (n=4), or PBS only (n=4) for 30 min prior to the final anastomosis. Animals survived for 7 days at which time flow probe analysis was performed and the outflow tract was harvested and histology was performed. FIG. 7 contains representative stained sections showing the intimal hyperplasia of the venous outflow tract associated with this model and data demonstrating that βARKct expression significantly attenuates this process. In addition to the histological data, average flow through Adeno-βARKct treated grafts (187±14 ml/min) was statistically greater than flow through control animals (96±6 ml/min, p<0.05) (t-test).

In the 28 day study, failure of the Gore-tex grafts (AV shunts) was observed when analyzed at weekly intervals via intra-vascular sonogram (IVS) to measure flow-rate through the shunt. At the end of one month, only 5 of the 12 grafts were open (4 each of PBS, EV and Adeno-βARKct treated). After determining the treatment groups at the end of the study, it was found that all 4 βARKct-treated pigs AV shunts were open and only one of the 8 control grafts were patent. This result is shown in FIG. 8 plotted as a survival (patency rate) curve. The mean patency time for the two control groups was <20 days.

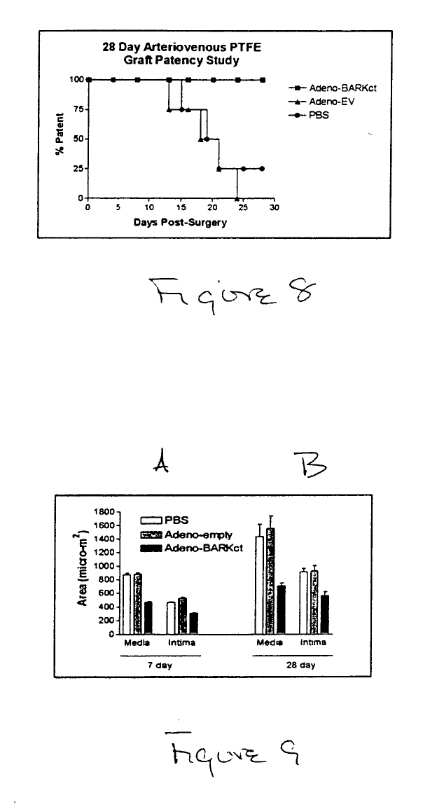

At the end of the 28 day study, sections of the outflow tract were studied histologically to examine medial and intimal thickness and neointimal hyperplasia and the results of medial and intimal area are shown in FIG. 9. The 28 day results are shown along with the 7 day results and demonstrate significant decreases in both medial and intimal growth of the Gore-tex AV shunts (grafts) treated with Adeno-βARKct. These results demonstrate that Adeno-βARKct treatment results in a significant increase in the patency of Gore-tex AV shunts and that medial and intimal areas are significantly decreased.

All documents cited above are hereby incorporated in their entirety by reference.

One skilled in the art will appreciate from a reading of this disclosure that various changes in form and detail can be made without departing from the true scope of the invention.

What is claimed is:

1. A method of inhibiting proliferation of vascular smooth muscle cells at the venous outflow tract of an arteriovenous shunt or fistula comprising introducing into said cells an inhibitor of Gβγ-mediated processes in an amount and under conditions such that said inhibition of vascular smooth muscle cell proliferation is effected.

2. The method according to claim 1 wherein said inhibitor is a polypeptide.

3. The method according to claim 2 wherein said polypeptide inhibits binding of β adrenergic receptor kinase (βARK) to Gβγ.

4. The method according to claim 3 wherein said polypeptide corresponds to the βARK Gβγ binding domain.

5. The method according to claim 4 wherein said polypeptide has the amino acid sequence set forth in SEQ ID NO:2 or portion thereof that includes at least amino acids 150-177 of said SEQ ID NO:2 sequence.

6. The method according to claim 2 wherein a nucleic acid sequence encoding said polypeptide is introduced into said cells under conditions such that said nucleic acid is expressed and said polypeptide is thereby produced.

7. The method according to claim 6 wherein said polypeptide inhibits binding of β adrenergic receptor kinase (βARK) to Gβγ.

8. The method according to claim 7 wherein said polypeptide corresponds to the βARK Gγβ binding domain.

9. The method according to claim 8 wherein said polypeptide has the amino acid sequence set forth in SEQ ID NO:2 or portion thereof that includes at least amino acids 150-177 of said SEQ ID NO:2 sequence.

10. The method according to claim 6 wherein said nucleic acid is present in a vector.

11. The method according to claim 10 wherein said vector is a viral vector.

12. The method according to claim 11 wherein said vector is an adenoviral vector.

13. The method according to claim 1 wherein said Gβγ-mediated process is Gβγ-mediated proliferative signaling.

14. A method of manufacturing a stent or shunt comprising bonding to, or incorporating into, a stent or shunt an agent that inhibits proliferation of cells at risk of proliferation upon implantation into a patient of said stent or shunt, said agent being an inhibitor of Gβγ-mediated processes.

15. The method according to claim 1 wherein said cells are vascular smooth muscle cells.
